(19) United States
(12) Patent Application Publication
(10) Pub. No.: US 2008/0077005 A1
(43) Pub. Date: Mar. 27, 2008

(54) System and Method for Multimodality Breast Imaging

(76) Inventors: Cameron Anthony Piron, Toronto (CA); Christopher Alexander Luginbuhl, Toronto (CA); Jae Koul Kim, Tucson, AZ (US)

Correspondence Address: QUARLES & BRADY LLP, 411 E. WISCONSIN AVENUE, SUITE 2040, MILWAUKEE, WI, 53202-4497, US

(21) Appl. No.: 11/949,445

(22) Filed: Dec. 3, 2007

Related U.S. Application Data
(63) Continuation-in-part of application No. 10/916,738, filed on Aug. 12, 2004.
(60) Provisional application No. 60/872,345, filed on Dec. 1, 2006.

(51) Int. Cl. A61B 5/055

(52) U.S. Cl. 600/411

(57) ABSTRACT

An imaging acquisition assembly for acquiring images using electromagnetic radiation and an imaging station are provided for use in imaging a breast using both magnetic resonance imaging techniques and electromagnetic radiation techniques. The acquisition assembly includes an elevating platform to which an electromagnetic source and detector are mounted, rendering the source and detector selectively positionable around a breast for electromagnetic imaging such as XRM or PET. The source and detector can be mounted to a rotatable disk to allow for imaging the breast from various angles, and can also be offset from vertical to allow other imaging configurations.

### CROSS REFERENCE TO RELATED APPLICATIONS

This application is a continuation-in-part of U.S. patent application Ser. No. 10/916,738, filed Aug. 12, 2004, and claims the benefit of U.S. Provisional Patent Application No. 60/872,345, filed Dec. 1, 2006.

### BACKGROUND

X-ray mammography (XRM) is the most commonly used imaging modality for the detection and diagnosis of breast cancer and is currently the only modality used for screening in the general population. However, X-ray mammography has some shortcomings with respect to sensitivity and specificity, particularly in younger women and in women with dense breast tissue. This deficiency has led to the investigation of alternative imaging modalities for breast imaging, including: ultrasound (US), magnetic resonance imaging (MRI), nuclear medicine, X-ray computed tomography (CT), positron emission tomography (PET), single-photon-emission computed tomography (SPECT), near-infrared tomography (NIRT), and optical imaging techniques including optical computed tomography (OCT), for example.

Of these modalities, MRI has proven most useful for screening younger and high-risk women for whom X-ray mammography is least reliable. MRI obtains three-dimensional images of the breast with excellent soft tissue contrast, providing a detailed depiction of the breast anatomy. The use of MRI contrast agents to increase the image intensity of highly vascularized tissue enhances visualization of malignant lesions against the darker backdrop of normal parenchymal tissue. Recent multicenter screening studies of high-risk women reported detection sensitivities of 93-100% by adding MRI to XRM compared to 25-59% using XRM alone. Evidence-based guidelines have recently been adopted by the American Cancer Society recommending annual MRI screening of women at high risk for developing breast cancer.

While the reported specificity of breast MRI has increased as image quality has improved and radiologists' experience with the technique has grown, however, it is still typically lower than the sensitivity, ranging from 81% to 99% in the high-risk screening studies. Focal enhancement of normal breast parenchyma, benign lesions, and benign proliferative changes may mimic an enhancing malignancy, resulting in false-positive MR examinations. The use of additional imaging modalities in combination with MRI, therefore, is desirable to provide complimentary information resulting in improved diagnostic specificity.

Although software techniques are known for combining imaging modalities using image registration and image fusion algorithms, the inhomogeneous, anisotropic nature of the soft tissue within the breast, its inherent non-rigid body behavior, and temporal changes of the breast tissue with menstrual cycle, make breast image registration and fusion a particularly challenging task. These problems are complicated by the different postures required for the patient during imaging with different modalities.

To meet these needs, systems which maintain patient positioning during ultrasound and PET or CT scans are known. U.S. Pat. Nos. 6,846,289, and 6,102,866 for example, disclose an integrated stereotactic XRM and US scanning system for imaging a standing patient's breasts. An integrated system for performing both PET and XRM scans with the patient upright has also been introduced in the commercial market by Naviscan PET Systems (Rockville, Md., USA). Commercial whole-body PET/CT systems that image patients lying on a table inside a cylindrical bore are also known. These systems obtain functional PET and anatomical CT images in a single session and co-register the multi-modality data. While effective in increasing the specificity as compares to single PET or US scans, however, these types of images are generally less effective than MRI scans.

Systems that combine MRI with other imaging modalities are also known. For example, a system that images the breast using MRI and NIRT simultaneously is known. This system incorporates a patient support structure including an integrated RF coil for MR imaging and a ring of photomultiplier tube (PMT) detectors for NIRT. Additionally, U.S. Patent Application 2005/0080333, published Apr. 14, 2005, discloses a system for integrating MRI and ultrasound examination of the breast. Here, the ultrasound examination is conducted outside of the MRI system while the patient is maintained in the same position throughout both examinations.

A method for acquiring images during both an MRI exam and an ancillary imaging exam based on detection of electromagnetic radiation such as XRM, PET, nuclear medicine imaging, or other modalities is also desirable. However, integrating MRI with electromagnetic radiation imaging presents a number of unique problems. For example, the limited space available inside the bore of a cylindrical MRI system makes it difficult to integrate MRI with larger imaging elements, such as two-dimensional detector array panels. Additionally, the detector elements used in modalities based on electromagnetic radiation are typically constructed of materials that are incompatible with MRI, or that create suboptimal imaging conditions in the breast for MRI. The present invention addresses these issues.

### SUMMARY OF THE INVENTION

In one aspect, the present invention provides a method for acquiring breast images using magnetic resonance imaging and an ancillary imaging method based on detecting electromagnetic radiation. The method comprises positioning a patient in a prone position on a patient support structure, immobilizing the patient's breast using an immobilization frame, and moving the patient and the patient support structure into a magnetic resonance imaging system. Magnetic resonance image of the patient's breast can then be acquired. The patient can then be removed from the magnetic resonance imaging system, and a detector element for electromagnetic radiation to the patient support structure and adjacent the patient's breast can be provided to acquire an electromagnetic radiation image of the breast. The magnetic resonance image and the electromagnetic radiation image can then be co-registered.

In another aspect, the present invention provides an imaging station. The station includes a patient bed including a patient support structure having an opening positioned to allow a breast of the patient to hang pendant through the opening, and an immobilization frame coupled to the patient support for immobilizing and compressing the breast of the patient. The station further includes a support component sized and dimensioned to receive the patient bed. The support component includes an upper surface for supporting the patient bed, and an opening provided in the support component defining an interventional volume for providing access to the breast of the patient in the immobilization frames from a plurality of angles. An elevatable platform is provided in the interventional volume, and an imaging source and a detector component adapted to acquire electromagnetic images of the breast are coupled to the elevatable platform, such that, when the patient bed is positioned on the upper surface of the support component with the patient support structure aligned over the interventional volume, the elevatable platform is selectively raised to position the imaging source and detector components on opposing sides of the breast in the immobilization frame, enabling acquisition of an electromagnetic image of the breast.

In still another aspect of the invention, an image acquisition assembly is provided for acquiring images of an immobilized breast using electromagnetic radiation. The image acquisition assembly comprises an elevatable platform, an electromagnetic source coupled to a first side of the platform and extending above the platform, and an electromagnetic detection device coupled to the opposing side of the platform and extending above the platform in opposition to the electromagnetic source and spaced a distance from the source selected to allow a breast of a patient to be selectively positioned between the electromagnetic source and the electromagnetic detection device. In use, the elevatable platform can be selectively raised to position the source and detector on opposing sides of the breast for the acquisition of electromagnetic images.

These and other aspects of the invention will become apparent from the following description. In the description, reference is made to the accompanying drawings which form a part hereof, and in which there is shown a preferred embodiment of the invention. Such embodiment does not necessarily represent the full scope of the invention and reference is made therefore, to the claims herein for interpreting the scope of the invention.

### BRIEF DESCRIPTION OF THE DRAWINGS

Embodiments are illustrated by way of example and not limitation in the figures of the accompanying drawings, in which like reference numerals indicate corresponding, analogous or similar elements, and in which:

FIG. 1A is a schematic diagram of a patient lying prone on a patient bed that includes a patient support structure, with the patient support structure supported atop a patient transport in accordance with one embodiment of the invention.

FIG. 1B is an illustration of the patient transport of FIG. 1A docked to an ancillary imaging station.

FIG. 1C is an illustration of the patient support of FIG. 1A translated onto a support component of the ancillary imaging station with the patient's breasts positioned over in interventional area including a raising/lowering platform for raising and lowering imaging source/detector components until they are appropriately positioned for imaging the breast(s).

FIG. 2A is a schematic diagram illustrating a compression frame compressing a single breast in the medial-lateral direction and including modular inserts housing RF coil elements 207 in the compression frames 206.

FIG. 2B is a schematic diagram illustrating the compression frame of FIG. 2A with the RF coil components removed, and with ancillary imaging source/detector components positioned immediately outside of the compression frames on either side of a single breast of interest.

FIG. 3A is a schematic diagram illustrating imaging source/detector components for tomographic imaging mounted on a disk that can be rotated around an axis to collect imaging data at multiple angles around a breast in the compression frame of FIG. 2A.

FIG. 3B is a top view of the schematic diagram of FIG. 3A

FIG. 3C is a schematic diagram of the disk of FIG. 3A with a non-vertical axis of rotation.

FIG. 4A is a side view of a compression frame assembly that includes two pairs of compression frames to compress two breasts simultaneously and RF coil elements inserted in the compression frames 406 for imaging both breasts using MRI.

FIG. 4B shows the compression frame assembly of FIG. 4A with the RF coil elements removed, and two pairs of ancillary imaging source/detector elements positioned immediately outside of the compression frames on either side of each breast.

FIG. 4C shows the compression frame assembly of FIG. 4A including two corresponding pairs of imaging elements positioned outside of the compression frames and mounted on disks, each of which disks rotates around a separate vertical axis.

FIG. 5 shows an embodiment in which an ancillary imaging device is located next to a patient transport with a large interventional volume; and

FIG. 6 shows an additional alternative embodiment in which imaging components are mounted directly on a patient transport during an ancillary imaging procedure.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION OF THE PREFERRED EMBODIMENT

In the following detailed description, numerous specific details are set forth in order to provide a thorough understanding of embodiments. However it will be understood by those of ordinary skill in the art that the embodiments may be practiced without these specific details. In other instances, well-known methods, procedures, components and circuits have not been described in detail so as not to obscure the embodiments.

By maintaining the patient in the same position during MR imaging and an ancillary imaging examination conducted outside the MRI suite, MR imaging may be combined with imaging modalities based on the detection of electromagnetic radiation such as XRM, PET, and others such that the position and conformation of the breast are matched in these multiple sets of images. According to an embodiment of the invention, a patient support structure that supports the entire patient with apertures for the breasts may be used for MR breast imaging. RF coil elements positioned around the patient's breasts and coupled to the patient support structure may be used to perform MRI. Appropriate patient support structures and coil elements for this application are disclosed, for example, in U.S. Patent Application 2005/0080333, published Apr. 14, 2005, which is hereby incorporated herein by reference for its description of these devices.

After MR imaging is completed, the RF coil elements may be removed, and the patient may be transported on a patient transport to an area outside the MRI suite for an ancillary imaging examination, while the patient is maintained in the patient support structure. The patient and patient support structure may then be positioned on an appropriate support or supports including an open interventional access area or interventional volume such that enough access is provided to the patient's breasts under the patient support structure to enable an ancillary imaging procedure to be performed without repositioning the patient relative to the patient support structure. This may be accomplished in multiple different ways, including for example, by using: 1) an ancillary imaging station to which the patient transporter may be docked without disturbing the patient, and to which the patient support structure and the patient may be transferred once the patient transporter is docked; 2) a patient transport that has enough space under the patient support structure to temporarily accommodate an ancillary imaging device; or 3) ancillary imaging equipment integrated into the patient transporter and/or the patient support structure.

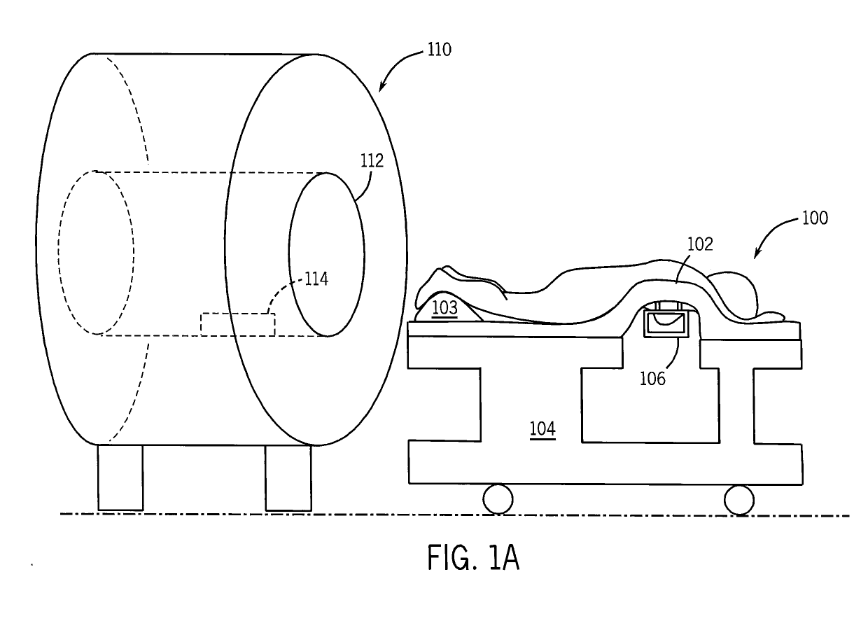

FIGS. 1A-1C are schematic diagrams of a patient positioned on a patient bed for use with an embodiment of the invention. FIG. 1A shows a patient bed 100 that includes a patient support structure 102 shown supported atop a patient transport 104 with the patient lying prone on the patient support structure 102. The patient's feet are shown supported using a cushion 103. The patient transport 104 is shown positioned for docking to a MRI system 110. The patient transport 104 docks to the MRI system 110 such that the patient support structure 102 may be translated into the bore 112 of the MRI system 110 for MR imaging, as described more fully in U.S. Patent Application 2005/0080333, incorporated herein by reference for its description of such devices. The patient support structure 102 attaches to a moving station 114 in the bore 112 of the MRI system 110. The patient support structure 102 has coupled to it immobilization frames 106 for providing mild compression to a breast to immobilize it during MR imaging. The immobilization frames 106 are shown positioned to provide medial-lateral compression, with pairs of immobilization frames 106 compressing each breast from the medial and lateral aspects. These devices are also disclosed in U.S. Patent Application 2005/008033, hereby incorporated by reference for its description of these devices. The immobilization frames 106 are preferably made of a material that is both MRI-compatible and is transparent to the wavelength of electromagnetic radiation used for the ancillary imaging exam. For example, if the ancillary imaging exam is to be XRM, the immobilization frames 106 are preferably constructed from x-ray transparent material. Alternatively, the immobilization frames 106 may be constructed from a material whose attenuation may be characterized and corrected for in the resulting images as is known in the art. Examples of suitable materials include most plastics including but not limited to: polycarbonate, acrylic, polyetheretherketone (PEEK), fiberglass, fiberglass composite materials, Kevlar® etc.

FIG. 1B shows the patient transport 104 docked to an exemplary ancillary imaging station 120. The ancillary imaging station comprises a support component 122 including an upper surface 123 for receiving the patient support structure, and an open imaging/interventional area or volume 125. The open imaging/interventional area or volume 125 is sized and dimensioned to allow access to the area of interest, here specifically the breasts, of the patient supported on the upper surface 123 of the imaging station 120 from both sides. A raising/lowering platform 124 which can be, for example, driven up and down either manually or by a motor, hydraulic lift, or other devices, is provided in the interventional volume 125, and imaging detector components 126 are coupled to the platform 124. Alternatively, the imaging detector components 126 may be supported by an arm or other support member (not shown).

In use, the patient support structure 102 is positioned on the upper surface 123 of the support component 122 of the ancillary imaging station 120, until the patient's breasts are positioned over the imaging/interventional volume 125, and aligned over the platform 124 and imaging components 126. When so positioned, the raising/lowering platform 124 raises imaging detector components 126 until they are appropriately positioned adjacent the immobilization frames 106 for imaging the breast(s) as shown in FIG. 1C. Preferably, the immobilization frames 106 remain in the same position throughout MR imaging and the ancillary imaging examination, maintaining the breast(s) in the same position and conformation during both examinations. Alternatively, the breast(s) may be imaged without compression for either imaging exam, i.e. without the immobilization frames 106. In a further alternative embodiment, the breasts may be compressed to a different compression thickness for the ancillary imaging modality compared to the MR imaging. While the immobilization frames 106 are shown providing compression to the breasts in the medial-lateral direction, they may be alternatively configured to provide compression to the breasts in a cranial-caudal direction, or an oblique medial-lateral, or an oblique cranial-caudal direction. Guiderails (not shown) may be used to constrain translation of the patient support structure 102 onto the ancillary imaging station 120. Alternatively, other components or approaches may be substituted, including for example guide pins, fitted pins, wheels and tracks, etc. Electronics and other components necessarily associated with the ancillary imaging system 120 as known in the art are not shown in order to better illustrate the location of the ancillary imaging components relative to the immobilization frames.

While FIG. 1 illustrates a patient transport 104 that docks to a MRI system, alternative embodiments are anticipated wherein a patient transport is used that does not have the capability of docking directly to a MRI system, but rather, is able to accept a patient support structure from the MRI system without repositioning of the patient. While some commercial MRI systems have capabilities for allowing the docking of a patient transport, others do not. In the latter case, a patient transport may be used that can be positioned next to or end-to-end with the patient support structure such that the patient support structure may be translated from the MRI system to the patient transport without repositioning the patient.

The imaging detector components 126 may be a single detector, as is used for example in XRM or internal reflection microscopy (IRM). Non-limiting examples for the detector are a film sensitive to the wavelength of the source radiation, or a digital detector panel such as a charge-coupled device, an array of semiconductor pixel detectors, or an amorphous semiconductor detector plate for example. Alternatively, the imaging detector components 126 may be a pair of detectors, for example gamma emission scintillation detectors such as cadmium-zinc-telluride (CZT) detectors as would be used for electromagnetic radiation sources internal to the body as for example, in PET or SPECT imaging. The imaging detector components 126 may alternatively comprise a ring of detectors, or any other configuration of multiple detectors.

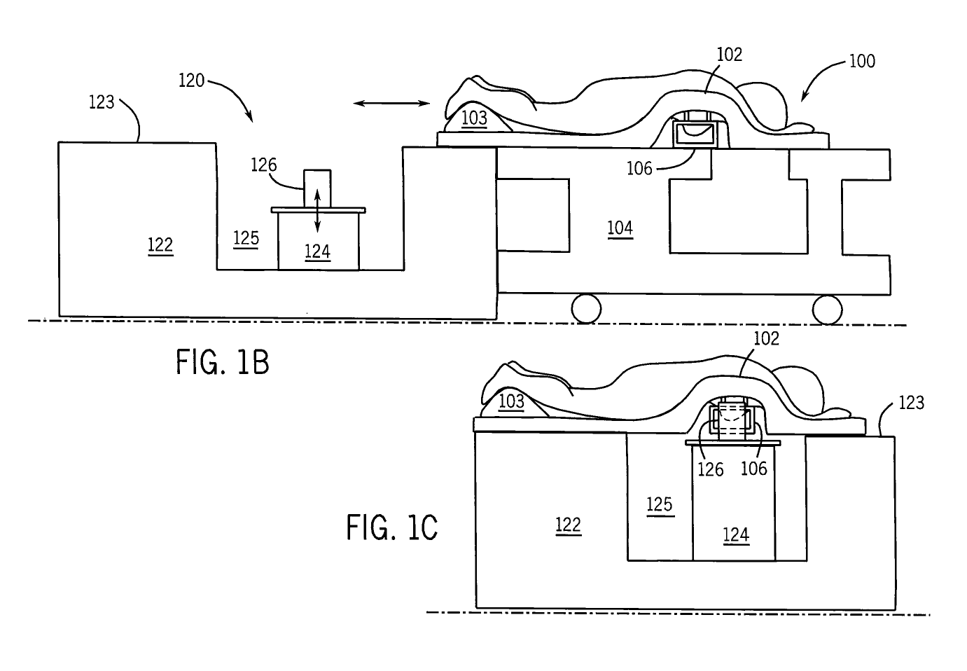

FIGS. 2A-2B illustrate a breast compressed between two compression frames for single projection imaging in accordance with an embodiment of the invention. FIG. 2A shows a single breast compressed in the medial-lateral direction using radiolucent compression frames 206. Modular inserts housing RF coil elements 207 are shown inserted in the compression frames 206. The contralateral breast is shown compressed near the chest wall using an obliqued horizontal breast support 208. As shown in FIG. 2B, the RF coil components 207 have been removed, and two ancillary imaging detector components 226 have been positioned immediately outside of the compression frames 206, on either side of a single breast of interest. Alternatively, the ancillary imaging detector components 226 may be mounted in the compression frames 206 (this embodiment not shown). A single detector component as would be used for example, in XRM may be alternatively used. A radiation source (not shown) such as a conventional x-ray tube may positioned away from the breast at an appropriate distance and orientation to create a mammographic image at the detector component 226.

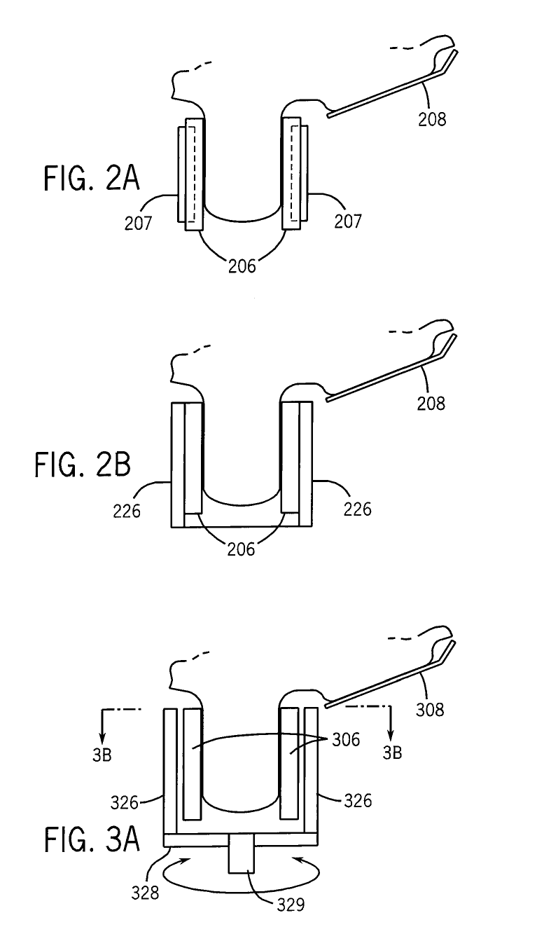

FIGS. 3A-3C illustrate a breast compressed between two compression frames 306 for tomographic imaging techniques such as PET, SPECT or x-ray tomography in which multiple projections are acquired as is known in the art. FIG. 3A and FIG. 3B show imaging detector components 326 mounted on a disk 328 that can be rotated around an axis 329, either manually or mechanically by means of a motor or other device, to collect imaging data at multiple angles around a breast. As shown in FIG. 3C, the axis of rotation 329 for the disk 328 is not constrained to be vertical, but can be angled at various orientations. The axis 329, therefore, may be configured to allow multiple axes of rotation for the disk 328. The contralateral breast is shown supported by an obliqued horizontal breast support 308. For x-ray tomography, a radiation source (not shown) positioned at an appropriate distance and orientation to create an image at the detector component 226 may be adjustable on an arc about the breast opposing the detector component 226 such that multiple projection images of the breast may be acquired.

In an exemplary embodiment, the ancillary imaging station may be a prone mammography/biopsy table as is described for example in U.S. Pat. No. 5,776,062, hereby incorporated herein by reference for its description of such a device. The patient support structure may replace the patient table conventionally associated with the prone mammography/biopsy table. Functionality for adjusting the inclination of the patient support structure from the horizontal position may be included, allowing medical personnel more working space in which to maneuver imaging or biopsy components below the patient. Alternatively, the ancillary imaging station may incorporate PET technology for breast imaging similar to the technology marketed for upright breast imaging as the PEM Flex Solo imaging system by Naviscan PET systems.

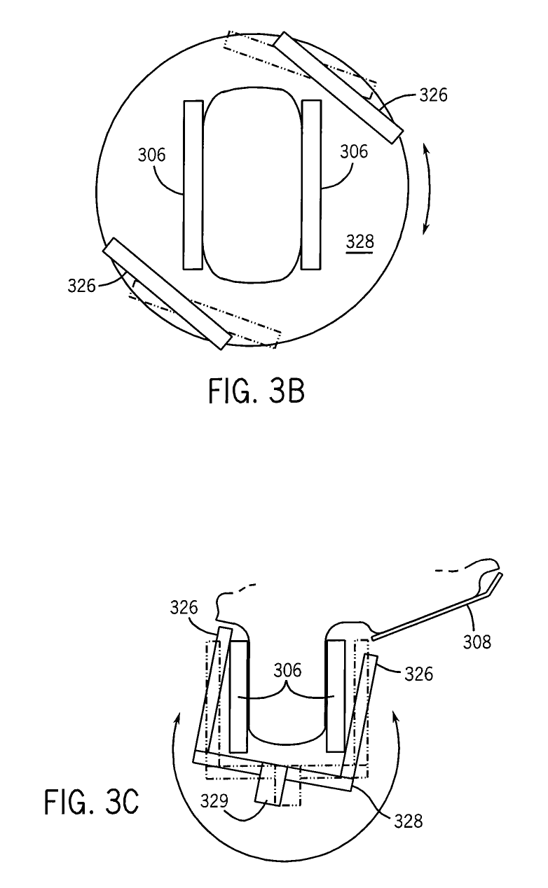

FIGS. 4A-4C illustrate a compression frame assembly for compressing both breasts for bilateral imaging. In FIG. 4A, two pairs of compression frames 406 are used to compress the breasts and RF coil elements 407 are inserted in the compression frames 406 for imaging both breasts using MRI. FIG. 4B shows the compression frames 406 with the RF coil elements 407 removed. Two pairs of ancillary imaging source/detector elements 426 are positioned immediately outside of the compression frames 406 on either side of each breast. FIG. 4C shows two pairs of ancillary imaging elements 426 positioned outside of the compression frames 406 and mounted on two disks 428, each of which disks rotates around a separate vertical axis 429, 430.

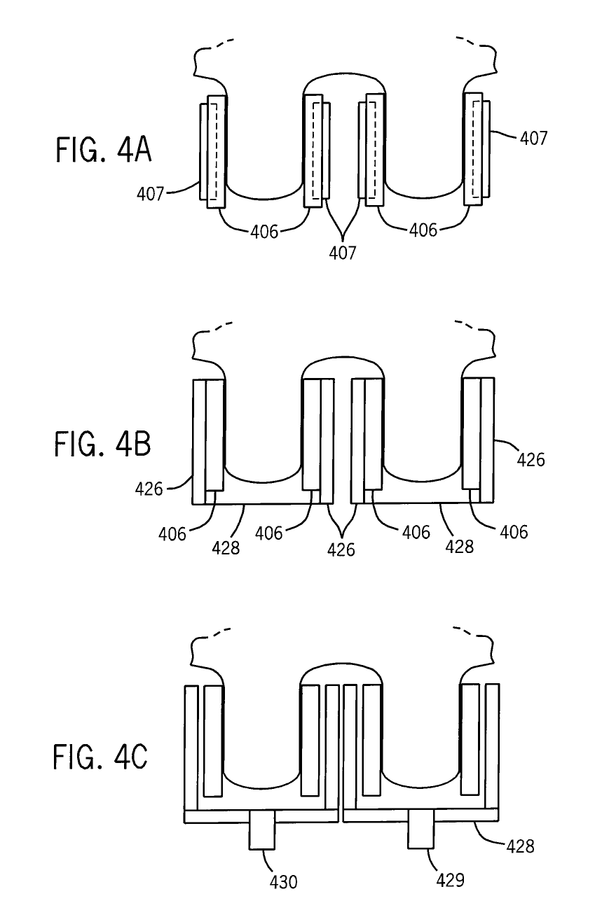

In an alternative exemplary embodiment, a patient is transported out of the MRI suite on a patient support structure that is supported by and coupled to the top of a dedicated patient transport. The dedicated patient transport includes a large interventional volume such that the area under the patient's breasts is open and accessible for additional imaging examinations and interventional procedures. FIG. 5 shows an embodiment in which a patient transport 522 with a large interventional volume 525 is positioned next to an ancillary imaging device 502. The patient is positioned on a patient support structure 520 atop the patient transport 522. The ancillary imaging device 502 includes a mechanical support 504 for ancillary imaging components 526. The mechanical support 504 allows the imaging components 506 to be positioned next to the patient's breasts. As for previous embodiments, the imaging components 506 may be any combination of radiation sources, detector panels or other detector elements.

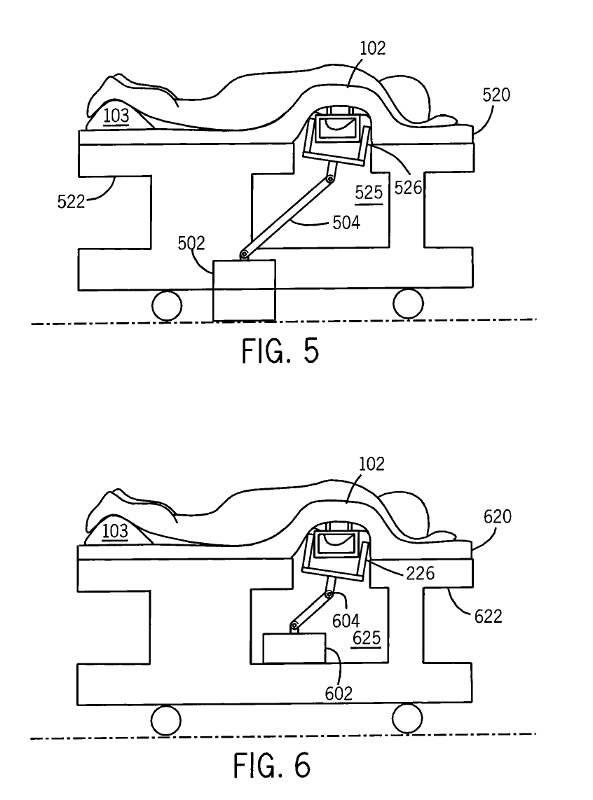

FIG. 6 shows an additional alternative embodiment in which the ancillary imaging device 602 and imaging components 626 are mounted directly in an interventional volume 625 of a patient transport 622 patient transport during the ancillary imaging procedure. These imaging components 626 may be mounted on the patient transport 622 for the ancillary imaging examination and removed for the MRI exam while the patient's breast(s) remain in the same position and conformation throughout. As described above with reference to FIG. 5, a mechanical arm 604 may be coupled to the imaging apparatus 626, and can be used to manipulate the position of the imaging components 626 relative to the breast of the patient. Although an articulated arm is shown here, various manual and automated mechanical devices for positioning, rotating, and otherwise changing the orientation of the imaging components 526 and 626 relative to the breast of the patient can also be used, as will be apparent to those of skill in the art.

Imaging the breast using MRI and an ancillary imaging modality while the breast is maintained in the same position and conformation improves image registration and image fusion. In addition, fiducial markers visible on both MR imaging and on the ancillary imaging modality may be used to facilitate registration of the images. The fiducial markers are preferably coupled to the immobilization frames proximal to the breast. The breast may be imaged in different conformations for MRI and for the ancillary modality, and the fiducial marker locations may be used to warp the images for co-registration.

Although the system is described above as acquiring an MR image of the breast first, and then acquiring an image using electromagnetic radiation, it will be apparent that the order of acquisition can also be reversed. Furthermore, more than one image set can be acquiring using one or more electromagnetic radiation modality or technique, and each of the sets of acquired images can be co-registered.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method for acquiring breast images using magnetic resonance imaging and an ancillary imaging method based on detecting electromagnetic radiation, comprising the steps of:
positioning a patient in a prone position on a patient support structure;
immobilizing the patient's breast using an immobilization frame;
moving the patient and the patient support structure into a magnetic resonance imaging system;
acquiring a magnetic resonance image of the patient's breast;
removing the patient and the patient support structure from the magnetic resonance imaging system;
coupling a detector element for electromagnetic radiation to the patient support structure and adjacent the patient's breast;
acquiring an electromagnetic radiation image of the breast; and
co-registering the magnetic resonance image and the electromagnetic radiation image.

2. The method of claim 1, further comprising the step of rotating the immobilization frame and acquiring images of the breast from at least two angles.

3. An imaging station comprising:
a patient bed including a patient support structure having an opening positioned to allow a breast of the patient to hang pendant through the opening;
an immobilization frame coupled to the patient support for immobilizing and compressing the breast of the patient;
a support component sized and dimensioned to receive the patient bed and including an upper surface for supporting the patient bed, and an opening provided in the support component defining an interventional volume for providing access to the breast of the patient in the immobilization frames from a plurality of angles;
an elevatable platform provided in the interventional volume; and
an imaging source and a detector component adapted to acquire electromagnetic images of the breast coupled to the elevatable platform, wherein when the patient bed is positioned on the upper surface of the support component with the patient support structure aligned over the interventional volume, the elevatable platform is selectively raised to position the imaging source and detector components on opposing sides of the breast in the immobilization frame, enabling acquisition of an electromagnetic image of the breast.

4. The imaging station of claim 3, further comprising a rotatable disk coupled to the elevatable platform.

5. The imaging station of claim 4, wherein the imaging source and the detector component are coupled to opposing sides of the rotatable disk, and the source and detector are rotatable to enable the acquisition of images from a plurality of angles.

6. The imaging station of claim 5, wherein the axis of rotation of the disk is selectively angled to provide at least one non-vertical angle of rotation.

7. The imaging station of claim 3, further comprising an RF coil element that is adapted to be selectively coupled to the immobilization frame for acquisition of MR images of the breast.

8. An image acquisition assembly for acquiring images using electromagnetic radiation, the image acquisition assembly comprising:
an elevatable platform;
an electromagnetic source coupled to a first side of the platform and extending above the platform; and
an electromagnetic detection device coupled to the opposing side of the platform and extending above the platform in opposition to the electromagnetic source and spaced a distance from the source selected to allow a breast of a patient to be selectively positioned between the electromagnetic source and the electromagnetic detection device;
wherein the elevatable platform can be selectively raised to position the source and detector on opposing sides of the breast for the acquisition of electromagnetic images.

9. The image acquisition assembly of claim 8, wherein the elevatable platform includes a disk mounted to a rotatable axis, wherein the source and detector are rotatable to a plurality of positions around the breast.

10. The image acquisition assembly of claim 9, wherein the rotatable axis is adapted to be adjusted to a plurality of angles.

11. The image acquisition assembly of claim 8, wherein the elevatable platform is coupled to a mechanical arm.
